(19) United States
(12) Patent Application Publication
(10) Pub. No.: US 2007/0218113 A1
(43) Pub. Date: Sep. 20, 2007

(54) Health bars and compositions for improving cardiovascular risk factors

(76) Inventors: Debra L. Miller, Palmyra, PA (US); Xiaoying Wang, Hummelstown, PA (US); Julia J. Watterson, Hummelstown, PA (US); Philip C. Ward, Palmyra, PA (US)

Correspondence Address: WILEY REIN LLP, 1776 K. STREET N.W., WASHINGTON, DC, 20006, US

(21) Appl. No.: 11/711,780

(22) Filed: Feb. 28, 2007

Related U.S. Application Data
(60) Provisional application No. 60/777,148, filed on Feb. 28, 2006.

(51) Int. Cl. A61K 47/00; A61K 36/48

(52) U.S. Cl. 424/439; 424/757

(57) ABSTRACT

The invention provides, in one aspect, a method of administering a product comprising beta glucan, soy protein, phytosterols, and an omega-3 fatty acid, such as one or more of DHA and EPA, to improve cardiovascular risk factors, such as LDL levels or total cholesterol levels. The amount of each of beta glucan, soy protein, phytosterols, and omega-3 fatty acids is selected from the amount providing the beneficial health effects associated with the health claims for these compositions or ingredients. Methods of making nutritional health bars and food ingredients that comprise the same compositions and ingredients in effective amounts that are shelf-stable are also disclosed.

### REFERENCE TO RELATED APPLICATIONS

This application claims full priority benefit of prior U.S. Provisional Application 60/777,148, filed Feb. 28, 2006, the entire contents of which are hereby incorporated by reference.

### FIELD OF THE INVENTION

The invention relates to food products and ingredients that comprise combinations of compounds that alone and together can measurably reduce cardiovascular risk factors, such as LDL cholesterol levels. The health bars, ingredients and products of the invention can be taken with a normal, weight-sustaining diet to reduce health risk factors. Advantageously, the health bars and products have an increased beneficial effect on those with high LDL cholesterol counts and those who are sensitive to insulin. Furthermore, the health bars, food ingredients, and methods of producing them provide solutions to the problem of adding sufficient amounts of omega-3 oils in food products without requiring encapsulation techniques.

### RELEVANCE OF THE INVENTION AND DESCRIPTION OF RELATED ART

Snack foods and breakfast bars have been produced that contain a desired ratio of fat to protein to carbohydrates. Many of these products are marketed as useful for particular diets that are high in protein. For example, snack bars and other high-protein products have been designed for diabetes mellitus patients and others on so-called restricted carbohydrate diets. High-protein bars have also been formulated for those attempting to gain muscle mass. However, the bars are typically used as a snack to either increase protein intake or reduce carbohydrate consumption. In addition, vitamin and other supplements are used in certain diets, again for specific conditions or consumers. The bars or supplements are not typically presented for use by the average consumer for general health maintenance or improvement of specific risk factors associated with cardiovascular health. In contrast, the present invention relates to a generally beneficial health bar or food ingredient that, among other benefits, reduces LDL cholesterol levels.

In addition, the use of soy protein for such bars is a known cost-effective way to enhance the nutritional profile. However, soy flour and other high protein soy ingredients absorb moisture which can lead to bar hardening over time. Just increasing the moisture content of the bar is undesirable because water can adversely effect certain ingredients, such as peanut products. Furthermore, the moist bars often have an undesirably short shelf life. Combinations with other healthy ingredients, such as one or more of beta-glucan, phytosterols, and omega-3 fatty acids such as docosahexaenoic acid (DHA) and eicosapentaenoic acid (EPA), can exacerbate the taste and stability problems. In fact, it is known in the art that omega-3 fatty acids impart undesirable off-flavors to food products.

From these deficiencies alone, one can conclude that new and improved methods for producing health bars and food ingredients with a higher protein content are needed. As noted above, the health bars, products and ingredients of the invention can also reduce cardiovascular risk factors when consumed with a normal diet.

### BRIEF SUMMARY OF THE INVENTION

The invention relates to compositions and methods of making or improving health bars or nutritional food products or ingredients. In one aspect, the bar or product contains approximately 50% energy as carbohydrate, 15% energy as protein, and 35% energy as total fat (50/15/35), which is about the ratio in the typical diet. A range of carbs/protein/fat values can, however, be selected and used. In a preferred aspect, the bar or product contains 40/30/30 (% of calories from carbs/protein/fat) and/or contains 35/10/35 (% of calories from total fat/ % of calories from saturated fats/ % total sugar by weight). In another aspect, the range of carbohydrate content can be from about 30 to about 55% or anything in between, the range of protein content can be about 15 to about 30% or anything in between, and the range of fat content can be about 25 to about 35% or anything in between. The production methods can be commercially scaled-up, even when the preferred ingredients of beta glucan, soy protein, phytosterols, and omega-3 fatty acids are used. In general, the omega-3 fatty acids used comprise one of or a combination of both of DHA and EPA, but other known omega-3 fatty acid compositions that contain an effective amount of beneficial compounds can be used. Advantageously, preferred compositions have a long shelf life and can be stored in hot transportation containers, if necessary, and maintain good taste characteristics after 6 months or more of total storage time.

In one aspect, the invention provides a nutritional bar or food product comprising a preferred group of components having beta glucan, soy protein, phytosterols, and omega-3 fatty acids. Each of these components are selected from one or more edible or food product sources that possess effective amounts of beneficial compounds as represented by health claims known in the art or available to one of skill in the art. In general, soy protein is present at about 10-30% by weight, phytosterols are present at about 1 to 2% by weight, omega-3 fatty acids are present at about 1% by weight, and beta glucan is present at about 5 to 6%. The bar or product can also contain a carbs/protein/fat content of about 40/30/30 on a percent of total calories basis. Especially preferred embodiments further comprise an antioxidant food ingredient, such as a cocoa product, a natural cocoa powder, cocoa extracts, or cocoa powder present at about 3% or more, and/or a cinnamon product and/or other strong antioxidant food ingredient or supplement. In additional embodiments, the bar or food product comprises one or more of cocoa liquor, fruit or fruit flavor, sunflower oil, fructose, insulin, or chromium picolinate. Plant sterols can be added or used, where plant sterols includes, without limiting to this specific list, phytosterols, phytosterol esters, phytostanols, phytostanol esters, and more particularly various positional isomers, stereoisomers, hydrogenated forms and/or phytostanol esters of the following non-limiting list of general plant sterols: sitosterol, campesterol, stigmasterol, spinosterol, taraxasterol, brassicasterol, desmosterol, chalinosterol, poriferasterol, clionasterol, avenosterol, and ergosterol. The above compounds or extracts containing them can be used alone or in any combination to provide two or more plant sterols or stanols for the products, health bars, or ingredients of the invention. Other nutritional supplements, vitamins, and/or minerals can also be included, in particular co-enzyme Q10.

In another embodiment, the invention comprises a method of producing a nutritional food product comprising preparing a dry ingredient mixture of protein crisps, nuggets and/or powder, soluble oat fiber, and dry milk; preparing a wet mixture of liquid fructose (about 90% in water), sunflower oil, phytosterols, and omega-3 fatty acids; mixing the dry and wet mixtures together with one or more flavors or flavoring ingredients, and forming the mixture into a final shape and size. Typically, the final product is extruded or slabbed, slit and cut, but other methods are known in the art and can be used. The final product can also be enrobed. Many enrobing compositions are known in the art and can be used. Preferred enrobing compositions include milk chocolate, dark chocolate, peanut butter coating, and white chocolate.

In another aspect, the invention provides a therapeutic or prophylactic method of treating one of many cardiovascular conditions, such as atherosclerosis, thrombosis, heart attack, stroke, or conditions characterized by an increased LDL cholesterol level. The method involves administering to a subject in need thereof, such as a subject with higher than average LDL cholesterol levels, a composition comprising: beta-glucan; soy protein; phytosterols; and one or more omega-3 fatty acids. As above, the composition can contain a carbs/protein/fat content of about 40/30/30% on an available energy basis. The method can be used in conjunction with a normal diet, or a diet that essentially maintains the current weight of the subject. The method can further comprise specifically monitoring the LDL cholesterol levels, total cholesterol levels, HDL cholesterol levels, and/or triglyceride levels of the subject.

In particularly preferred aspects, the bar or product and the methods of using it involve a composition that possesses a low glycemic index, as known in the art. Furthermore, the bar or product can be administered at least twice a day, such as at breakfast and dinner, and for periods of time, such as at least four weeks. These methods, as shown below, advantageously reduce LDL cholesterol levels in normal subjects. In preferred aspects, the bars are produced to or the methods designed to reduce LDL cholesterol level of the subject by about 5 mg/dl or more, or about 6 mg/dl or more, or about 7 mg/dl or more, or about 8 mg/dl or more. Further, the bars and methods can reduce total cholesterol levels by about 5 mg/dl or more, or about 6 mg/dl or more, or about 7 mg/dl or more. The products and methods of the invention can also be combined with other products and methods for reducing LDL or total cholesterol levels, for example one or more statins, fibrate drugs, resin-based therapies, which help keep the body from absorbing cholesterol in the gut, ezetimibe, and/or other drugs that interfere with the absorption of dietary cholesterol, and niacin.

While the preferred embodiments are given below and in the examples, the scope of the ingredients that can be selected for use should not be limited to those listed here. Other compositions containing the beneficial combinations, such as components with sufficient soy protein, beta glucans, phytosterols, and one or more omega-3 fatty acids, can be made and used according to this invention to generate the advantageous results described here. As known in the art, each of these components possess compounds that have a recognized ability to reduce cholesterol or improve cardiovascular health or risk factors. Accordingly, one of skill in the art is aware of available food products or ingredients that can supply an effective amount of one or more of soy protein, beta glucans, phytosterols, and omega-3 fatty acids to achieve the health claims associated with them. For example, the US Food and Drug Administration has approved or published information or health claims for at least the effective doses of soy protein, beta glucans, and phytosterols. In addition, recommended daily consumption levels of omega-3 fatty acids have been published by the Food and Nutrition Board and the American Heart Association

Throughout this disclosure, applicants refer to journal articles, patent documents, published references, web pages, and other sources of information. One skilled in the art can use the entire contents of any of the cited sources of information to make and use aspects of this invention. Each and every cited source of information is specifically incorporated herein by reference in its entirety. Portions of these sources may be included in this document as allowed or required. However, the meaning of any term or phrase specifically defined or explained in this disclosure shall not be modified by the content of any of the sources. The description and examples that follow are merely exemplary of the scope of this invention and content of this disclosure and do not limit the scope of the invention. In fact, one skilled in the art can devise and construct numerous modifications to the examples listed below without departing from the scope of this invention.

### BRIEF DESCRIPTION OF THE DRAWINGS

FIG. 1 depicts a graph of changes in blood LDL-cholesterol (LDL-C) as compared to the baseline represented by the ‘0’ change line. For subjects where the screened LDL-C is higher (on the bottom axis), the change following the administration of the health bar comprising beta-glucan, soy protein, phytosterols, and omega-3 fatty acids is most dramatic.

FIG. 2 depicts a graph of the changes in LDL-C against the insulin levels. As the subject is more sensitive to insulin and thus has a lower level of blood insulin on the bottom axis, there is a greater change in the LDL-C on the health bar diet.

FIG. 3 depicts a bar chart showing the average changes in LDL-C for each of three groupings (tertiles) of subjects. The tertile with the lowest levels of insulin and highest sensitivity to insulin record the greatest reductions in LDL-C levels.

FIG. 4 shows the statistical analysis of the results generated. The different error rates and statistical significance numbers are provided.

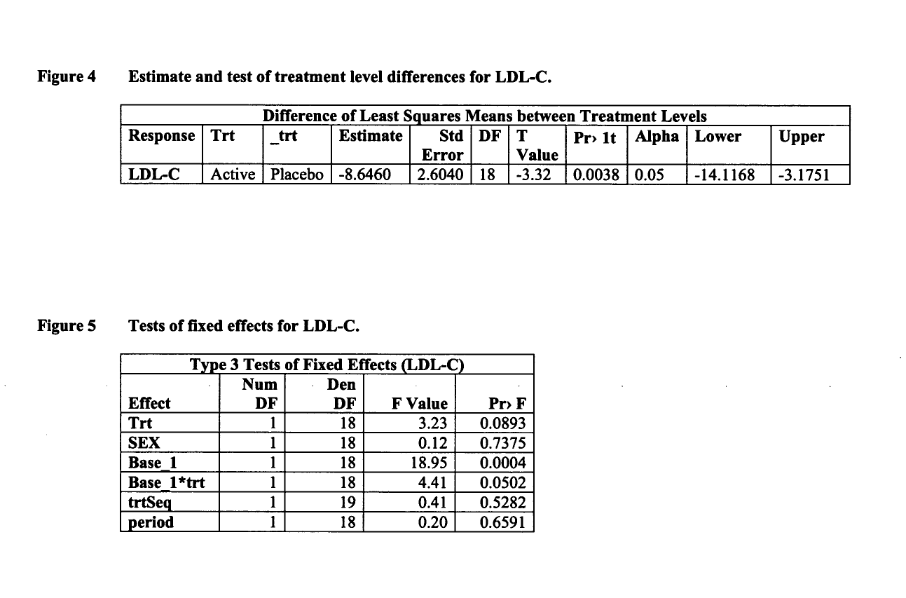

FIG. 5 represents a primary efficacy analysis. The primary response variable for efficacy is LDL-C (mg/dL) in FIG. 5. A mixed linear model is used in the evaluation of treatment efficacy. This model included as fixed effects gender (SEX), treatment level (trt), treatment period (period) and sequence (trtSeq). Average baseline LDL-C is included as a full covariate (including interaction with treatment). Subject (nested within treatment sequence) is included as random effects in the efficacy model. Response data employed in this analysis include the average of the two observations of LDL-C derived from non-consecutive days near the end of each diet period. The efficacy model estimates that the reduction in LDL-C attributable to the active treatment health bar administration group is 8.6±2.6 mg/dL relative to the placebo bar (p=0.0038 for the hypothesis of no difference vs. a two-sided alternative hypothesis).

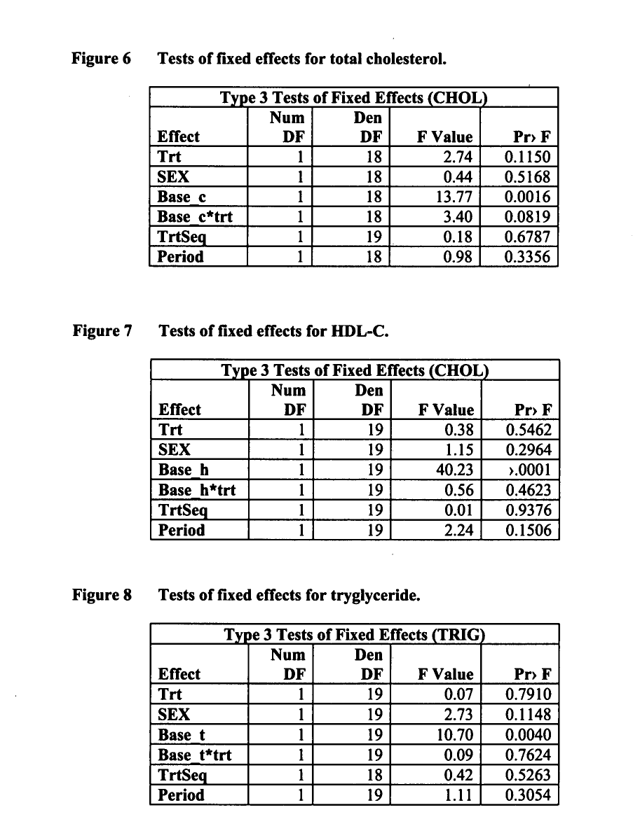

FIGS. 6-10 represent secondary analyses of response variables including total cholesterol (FIG. 6), HDL-C (FIG. 7), triglycerides (FIG. 8), and fasting serum glucose (FIG. 9) and insulin (FIG. 10). Analysis of secondary response measures is performed with models similar to the one cited for the primary efficacy analysis, except that average screening levels of the particular response variable were entered as the covariate. Screening values for insulin were not available and therefore, could not be included as a baseline covariate for that response. Serum concentration of triglycerides and insulin are natural log transformed prior to analysis. The evidence shows that the active level of treatment suppresses total cholesterol relative to the control (p=0.044). The test results incorporate the hypothesis of no mean difference between groups vs. a two-sided alternative hypothesis.

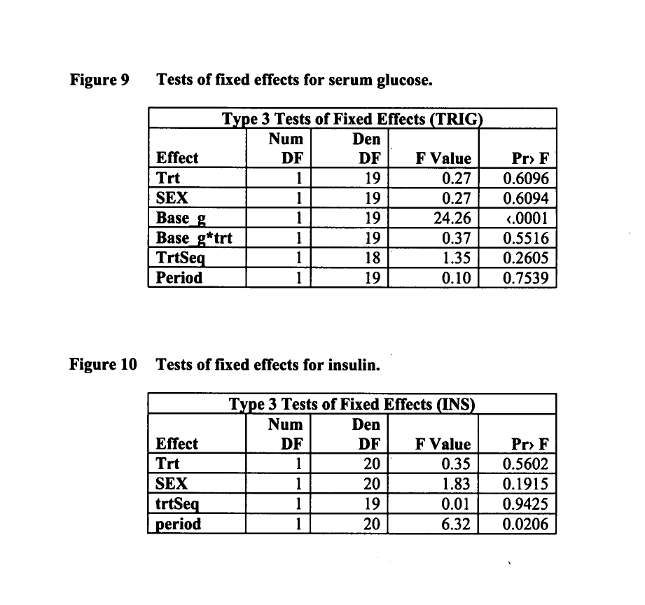

FIG. 11 shows the statistical analysis of the results generated and depicted in FIGS. 6-11. The different error rates and statistical significance numbers are provided.

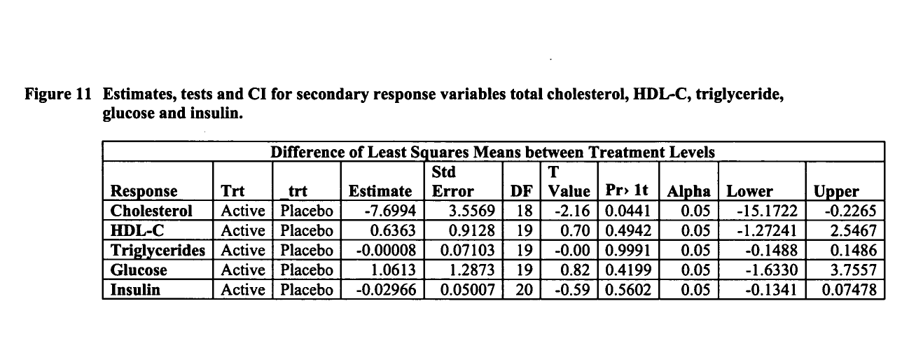

### DETAILED DESCRIPTION OF EXEMPLARY EMBODIMENTS

In a general aspect, the health bars and products of the invention can be designed with a content of about 40/30/30% carbs/protein/fat, however, as noted above, other ranges or specific levels of each can be selected and used. This bar further contains healthy ingredients designed to reduce one or more cardiovascular disease (CVD) risk factors. As shown below, a clinical trial can be designed to evaluate the efficacy of a nutritional bar intended to improve heart health and specifically to lower low density lipoprotein cholesterol (LDL-C) in healthy individuals who have moderately elevated LDL-C baseline. The nutritional bar is provided with an average American diet and was compared relative to a placebo bar. As noted above, the nutritional bar contained beta-glucan, soy protein, phytosterols, and omega-3 fatty acids, all known to improve markers of CVD risk. In preferred embodiments, one or both of DHA and EPA can be selected as the omega-3 fatty acids, and microalgal DHA in particular. Each of the components present in the nutritional bar independently lowers cholesterol, but the total reduction in cholesterol with all components together was unknown.

Furthermore, the preparation and storage of a bar with such components presents numerous challenges. In addition, producing a palatable taste and acceptable mouthfeel through typical storage periods is difficult for products with at least the omega-3 fatty acids component. Typically, microencapsulation or other encapsulation techniques are used with omega-3 fatty acids in these products and the invention does not require such encapsulation techniques to produce a stable product that does not become rancid after a week or less of storage. Rancidity can be a significant problem in products that contain omega-3 fatty acids. In fact, the products and health bars of the invention are stable up to six months. It was noted that under high heat conditions, bars containing and/or enrobed with a cocoa or chocolate product (containing flavanols) and those enrobed with a white chocolate or containing an added cinnamon and/or apple flavor had significantly less off-flavors associated with oxidation of omega-3 fatty acids compared to other coatings or other flavors. Sensory assessments of fishy off-flavors were conducted by a trained sensory panel, as known in the art. As explained below, these and other challenges have been met and a product and method to reduce CVD risk factors has been demonstrated.

In another aspect, the health bars of the invention utilize a high soy protein content. Alternatively, a combination of milk protein and soy protein can be used. In addition, a combination of different soy protein ingredients can be used to modify the characteristics of the bar. A combination of two soy proteins, for example a Solae soy protein isolate (#320) and a Solae partially hydrolyzed soy protein isolate (#313), at 16% and 7%, respectively, can be especially useful. Whey protein isolate at about 2 or 3%, or about 2.3%, can also be used. The combinations of different proteins can extend shelf life and improve taste characteristics.

In general, the protein used in the examples below is a 80% crisp soy nugget, which provides a good taste and texture and meets the effective amount of soy protein levels desired in the bar. A preferred water activity (Aw) of less than 0.5 is used in the bar to maintain a shelf life over 12 months. This also keeps water from migrating into the nuggets. At the same time, it is preferable to maintain a low glycemic index (GI) or load. Thus, traditional ingredients such as sugar and corn syrup should preferably be avoided because of the effect on GI. In other preferred embodiments, the levels of many low GI ingredients, such as polyols, that have a proper viscosity characteristic, sweetness and ability to control Aw could be used. However, care should be taken to avoid undesirable side effects associated with high levels of polyols.

In an especially preferred embodiment, the sweetener used is a liquid fructose (90% fructose) and it can be used in combination with glycerin at a ratio of about 16 to about 18:1. Total fructose levels can equal about 34-37%.

Many embodiments, bars and products of the invention may contain ingredients that are sensitive to water addition, namely chocolate liquor, peanut flour, or soy nuggets. This is because there is either an increase in product viscosity due to water interaction with peanut or cocoa solids or in the case of soy nuggets, a danger of over-saturation, making them soggy. As a result, a preferred process uses water with a minimal negative influence, which can be accomplished by handling all ingredients so that hydration and Aw were controlled. In one embodiment, this is done by proper order of addition of ingredients and assuring complete mixing. Another possible step is to add the sensitive ingredients last, when batch temperatures are reduced to under 110° F. This approach of efficiently adding chocolate liquor, peanut flour or soy nuggets at the end eliminates batch rheologies that are too high for practical extruding or slabbing and also helps to establish acceptable flavor or texture in the final product over a 12 month shelf life.

### EXAMPLES

Production of Exemplary Chocolate Health Bar

Many different flavors, flavoring agents, and ingredients can be selected for use with the basic bar containing beta-glucan, soy protein, phytosterols, and omega-3 fatty acids. Preferred embodiments can include food ingredients or supplements having strong antioxidant properties, such as cinnamon, a cocoa product, and/or cocoa powder. In this example, a chocolate-flavored center bar is used and then enrobed with a milk chocolate coating. The components employed that are associated with a particular health claim can be selected from many available in order to provide an effective amount. For example, the soy crisps or nuggets contain about 80% of an effective amount of the soy protein desired for the beneficial soy protein health claims. Similarly, the soy protein powder contains about 90% of the beneficial soy protein. Soluble oat fiber contains about 50% of the beneficial beta glucan desired for an effective amount of the health claims. Also, the beta glucan effective amount can be supplied by one or more of many food products or ingredients, including but not limited to whole oats, soluble oats, oat bran, rolled oats, psyllium husk, and other known or available compositions or extracts. The effective amount of phytosterols can be supplied by one or more of several plant-derived products or extracts, including but not limited to soy phytosterols, tall wood pulp oil, canola oil, plant sterols and/or stanols, and other known or available compositions or extracts. One of skill in the art is familiar with selecting and using a variety of food products or ingredients in order to supply an effective amount of one or more specific compositions or compounds having a beneficial health claim.

In the specific examples and recipes here, the components are selected for the desired taste, mouthfeel and shelf life characteristics. One of skill in the art is familiar with how to modify ingredient content to alter one or more of these characteristics, and thus these specific recipes should not be taken as a limitation of the scope of the compositions or bars made possible by this invention. In a preferred milk chocolate coated chocolate bar, the ingredients used are listed below.

| | |
|---|---|
| Final bar weight | 55 grams |

| Ingredient | Grams | Weight % |
|---|---|---|
| Coating | | |
| Milk Chocolate | 11.00 | 20 |
| Center | | |
| liquid fructose (90%) | 12.66 | 23.016 |
| soy crisps/nuggets | 9.24 | 16.8 |
| soy protein isolate | 7.51 | 13.648 |
| soluble oat fiber | 2.95 | 5.36 |
| cocoa liquor | 2.64 | 4.8 |
| cocoa powder | 1.76 | 3.2 |
| sunflower oil | 1.32 | 2.4 |
| nonfat dry milk | 1.32 | 2.4 |
| whey protein isolate | 1.32 | 2.4 |
| vitamin premix | 0.98 | 1.776 |
| glycerin | 0.88 | 1.6 |
| phytosterols (Plant sterol esters) | 0.75 | 1.368 |
| omega 3 oil | 0.57 | 1.04 |
| salt | 0.09 | 0.16 |
| lecithin | 0.02 | 0.032 |
| Total | 55.00 | 100.00 |

Center mixing procedure: Dry ingredient mixture—Dry blend soy protein powder, oat fiber, whey protein isolate, nonfat dry milk, vitamin premix, cocoa powder and salt in a double arm dough mixer. Wet ingredient mixture—Warm liquid fructose (90% in water) to 110 F in a mixing tank and add sunflower oil, glycerin, lecithin, plant sterol esters, omega 3 fatty acids (oil) and blend together. Add melted cocoa liquor to mixing tank with other wet ingredients and mix briefly. Add wet ingredient mixture to dry ingredient mixture and mix together until the mass comes together like a dough. Add soy crisps/nuggets and mix just until evenly dispersed.

Bar forming procedure: Center dough can be formed into bars by either extruding and cutting a long rope or by forming a continuous slab and then slitting and cutting to size.

Enrobing procedure: Bars are coated with chocolate using a chocolate enrober.

Production of Exemplary Apple-Cinnamon Health Bar

An apple-cinnamon flavored health bar center coated with white chocolate can be also be used. The ingredients used are listed below.

| | |
|---|---|
| Final bar weight | 55 grams |

| Ingredients | grams | % of bar |
|---|---|---|
| Coating | | |
| White chocolate | 11.00 | 20 |
| Center | | |
| Soy crisps/nuggets | 8.36 | 15.2 |
| Liquid fructose (90%) | 11.88 | 21.6 |
| Soy protein powder | 8.36 | 15.2 |
| sun flower oil | 3.17 | 5.76 |
| Soluble oat fiber | 2.95 | 5.36 |
| apple bits | 2.64 | 4.8 |
| Whey protein isolate | 1.98 | 3.6 |
| Nonfat dry milk | 1.01 | 1.84 |
| Vitamin premix | 0.98 | 1.776 |
| Glycerin | 0.88 | 1.6 |
| Plant sterol esters | 0.75 | 1.368 |
| Omega 3 oil | 0.57 | 1.04 |
| Cinnamon | 0.22 | 0.4 |
| apple/cinnamon flavor (Maxens) | 0.16 | 0.296 |
| Salt | 0.09 | 0.16 |
| Total | 55.00 | 100.00 |

Center mixing procedure: Dry ingredient mixture—dry blend the soy protein powder, oat fiber, whey protein isolate, nonfat dry milk, vitamin premix, cinnamon and salt in a double arm dough mixer. Wet ingredient mixture—warm liquid fructose (90% in water) to 110 F in a mixing tank and add sunflower oil, glycerin, phytosterols (plant sterol esters), and omega 3 fatty acids (oil) and flavor. Add wet ingredient mixture to dry ingredient mixture and mix together until the mass comes together like a dough. Add soy crisps/nuggets and apple bits, mix until evenly dispersed.

Bar forming procedure: Center dough can be formed into bars by either extruding and cutting a long rope or by forming a continuous slab and then slitting and cutting to size.

Enrobing procedure: Bars are coated with white chocolate using a chocolate enrober.

Testing of Health Bar on an Average Weight-maintenance Diet

Bars, such as the chocolate and apple/cinnamon bars above, can be used to test and monitor CVD risk factors. Other flavors, such as peanut butter, blueberry, and other fruits and nuts, can be selected. The study design was that of a two period crossover single-center study. Participants are randomized to treatment sequence, first the test or active product then a placebo bar, or first a placebo bar then a test or active bar. Treatment assignments are masked and the duration of each treatment phase was about five weeks.

A total of 22 subjects complete the study out of 23 original participants, seven were male and 16 female. There were 11 African-Americans, 10 Caucasians and 2 other. All were free of chronic disease and had LDL-cholesterol between 130-199 mg/dL based on the average of duplicate screening measures. No serious adverse events occurred during the study.

Baseline characteristics of the study participants (Table 1) are prepared from data, including selected anthropometric measures (height, weight, BMI—body mass index), demographic measures (gender, race, and age) and selected serum lab values (total cholesterol, LDL-C, HDL-C, triglycerides, serum glucose and insulin).

TABLE I
Baseline characteristics (n = 23).

| Variable | Mean ± SD |
|---|---|
| Height, cm | 165.3 ± 7.6 |
| Weight, kg | 78.5 ± 10.3 |
| BMI | 28.8 ± 3.7 |
| SBP, mm Hg | 115.4 ± 12.5 |
| DBP, mm Hg | 74.4 ± 9.0 |
| Resting HR | 70.6 ± 12.0 |
| Age, years | 52.6 ± 9.9 |
| Screening CHOL, mg/dl | 236 ± 20 |
| Screening TRIG, mg/dl | 124 ± 69 |
| Screening HDL, mg/dl | 58 ± 11 |
| Screening LDL, mg/dl | 154 ± 14 |
| Screening GLU, mg/dl | 98 ± 11 |

Each participant is fed an average American diet with calculated macronutrient composition, without the nutritional bars, as 15% energy protein, 50% energy carbohydrate, and 35% energy total fat. Normalized saturated, monounsaturated and polyunsaturated fatty acid values were 15%, 13%, and 7% energy, respectively. Calculated average dietary cholesterol was 96.6 mg/1000 kcal and calculated average dietary fiber was 5.7 g/1000 kcal.

Each participant received two (2) bars to consume each day, one with breakfast and one with dinner. Each bar provided approximately 220 kcal. The macronutrient content of the bars was 40% energy from carbohydrate and 30% energy each from fat and protein. The bars can be produced from the chocolate or apple/cinnamon examples above, or may contain other flavors such as blueberry or peanut butter. The nutritional bar contained (estimated):

| | Per serving | Daily amount of |
|---|---|---|
| Compound | (1 bar) | Health Claim |
| Beta-glucan (g) | 1.5 | 3 |
| Soy protein (g) | 12.5 | 25 |
| Phytosterols (g) | 0.65 | 1.3 |
| Omega 3 fatty acids (mg) | ˜150 | 300 |

Both diet interventions (test or active bar and placebo bar) were provided to participants for five weeks, each in a randomized crossover design. Upon completion of the entire protocol, each participant consumed both interventions of active bars and placebo bars.

Meals for participants are monitored or controlled during the period to allow for adjustment of energy intake and to become familiar with the diet routine. First, a pre-test meal is used with the placebo bar. Following successful completion of this pre-test period, participants were randomized to one of two possible sequences (active/placebo or placebo/active). The participants and all personnel involved in determining outcome variables were blinded with respect to the intervention. A short break was provided between the diet periods.

The participants were provided with all foods for the duration of the study and were encouraged to consume all foods provided. To assure consumption of the bars, the participants were required to consume one with breakfast and one with dinner. Participants recorded in a daily diary any deviations from the diet and the consumption of self-selected beverages.

A computerized randomization application is used to centrally randomize participants to a treatment sequence. The randomization was based upon a stochastic version of the Minimization Allocation method as presented by Pocock and Simon (Biometrics 31: 103-115 (1975). See also Taves, D R, Clin Pharmacol Ther 15: 443-453 (1974)). Treatment sequence allocation was such that the sequence selected was, with probability 0.75, the one that induced the smallest imbalance between treatment groups in a measure based on prognostic factors. Prognostic factors employed in the randomization were gender and LDL-C. For the purpose of randomization, screening LDL-C was dichotomized as follows: 130˜LDL-C<161 and 161<LDL-C≦199; units are mg/dL. A clinical database is used to maintain the unique identifier, date, time, and order of entry of the participant into the randomization table, along with the treatment sequence assignment, and the values of the prognostic variables employed in the randomization process (gender and LDL-C). The allocated treatment sequence for each study participant was transmitted to the PBRC metabolic kitchen; a blinding code was used to delineate treatment sequence (active/placebo bar or placebo/active bar).

Results collected for this study were total cholesterol, HDL-cholesterol, LDL-cholesterol (calculated), triglycerides, glucose, and insulin. However, the invention is not limited to the use of any particular assay of risk factor test and other available assays, including for example C-reative protein, can be selected and used. Assessments were made twice at the end of both diet periods on non-consecutive days, and were made in the morning prior to the breakfast meal and after a minimum 10-hour fast and 48-hour abstinence from alcohol. Assays were conducted at the end of the study in batches to include all of a given participant's samples at one time to minimize effects of inter-assay variation on outcomes.

LDL-C is used as a primary endpoint measure. The term “baseline” refers to observations derived from clinic visits and screening visits prior to the study. Fasting serum measurements made at baseline are averaged to yield a single baseline measure for each component (total cholesterol, HDL-C, LDL-C, triglycerides, glucose). Repeated tests can be used to replace original values. The “control” or “control condition” will reference the level of treatment that includes the placebo version of the nutrition bar.

The primary response variable is serum LDL-C, determined in a fasting state. Secondary response variables include total cholesterol, HDL-C, triglycerides, and fasting serum glucose and insulin. Serum concentration of triglycerides and insulin were log-transformed prior to analysis to improve distributional characteristics. However, the untransformed data were used as a covariate in inferential models and in generating tables of summary statistics.

Analysis of data from the study proceeded under intention-to-treat (ITT). That is, all participants randomized were considered for analysis according to their assigned treatment condition. Participants for whom at least one post-randomization observation (LDL-C lipid measure) had been obtained were to be included in the analysis set. Of the 23 participants randomized, 22 met this condition. Except as may be stated otherwise, statistical significance was defined relative to a 5% type 1 error rate (false positive rate) and tests related to pre-planned hypotheses are reported unadjusted for multiple comparisons.

Results

Dietary energy adjustments were made as needed in an effort to maintain weight within 1 kg of each participant's initial value. With the exception of one participant, weight was maintained within 1 kg, and as a group, did not deviate during the study (Table 2). The greatest change in weight from start to completion of the study was −1.6 kg.

TABLE 2
Body weight at start and end of each diet period (n = 22).

| Variable | Mean ± SD |
|---|---|
| Weight, kg, start period 1 | 78.0 10.2 |
| Weight, kg, end period 1 | 78.1 ± 10.1 |
| Weight, kg, start period 2 | 78.2 ± 10.4 |
| Weight, kg, end period 2 | 78.2 ± 10.5 |

Table 3 summarizes results of the endpoint measures and tests for mean treatment level. LDL-C was significantly lower with consumption of the nutritional bar relative to the placebo bar. Total cholesterol was also reduced.

TABLE 3
Cholesterol, triglyceride, glucose, and insulin response
to the nutritional bar and placebo bar (n = 22).

| Response | Nutritional Bar | Placebo Bar | Difference | P¹ |
|---|---|---|---|---|
| Cholesterol | 227.7 ± 21.4 | 235.8 ± 27.9 | −7.7 ± 3.6 | 0.0441 |
| (mg/dl) | | | | |
| LDL-C | 144.6 ± 18.5 | 153.4 ± 21.1 | −8.6 ± 2.6 | 0.0038 |
| (mg/dl) | | | | |
| HDL-C | 56.0 ± 11.6 | 55.3 ± 10.3 | 0.6 ± 0.9 | 0.4942 |
| (mg/dl) | | | | |
| Triglycerides | 135.4 ± 66.5 | 135.6 ± 63.7 | −0.0 ± 0.1 | 0.999 1 |
| (mg/dl) | | | | |
| Glucose | 100.7 ± 12.0 | 99.7 ± 10.8 | 1.1 ± 1.3 | 0.4199 |
| (mg/dl) | | | | |
| Insulin | 10.5 ± 4.3 | 11.0 ± 4.5 | −0.0 ± 0.1 | 0.5602 |
| (uU/ml) | | | | |

¹Least square means.

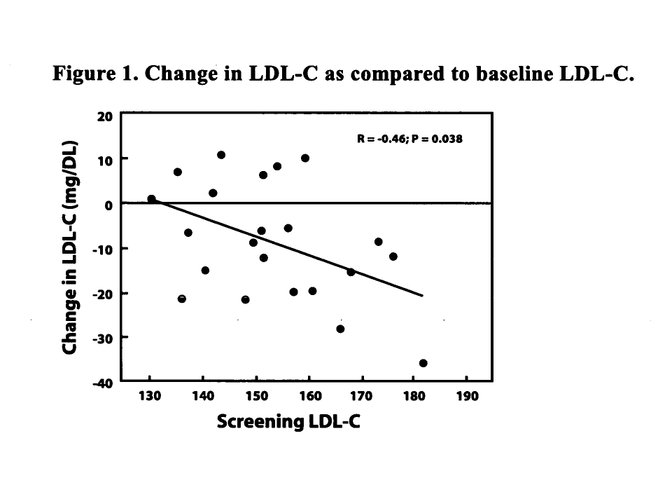

Surprisingly, the baseline LDL-C predicts the magnitude of the response to the health bar treatment. Those participants with higher LDL-C baseline had greater reductions or changes in LDL-C levels (FIG. 1). Baseline LDL-C was used as opposed to the control condition LDL-C to eliminate regression to the mean effects.

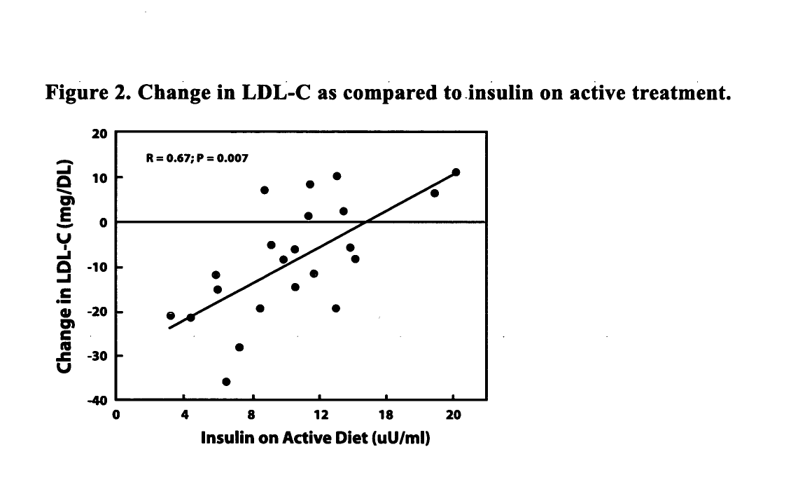

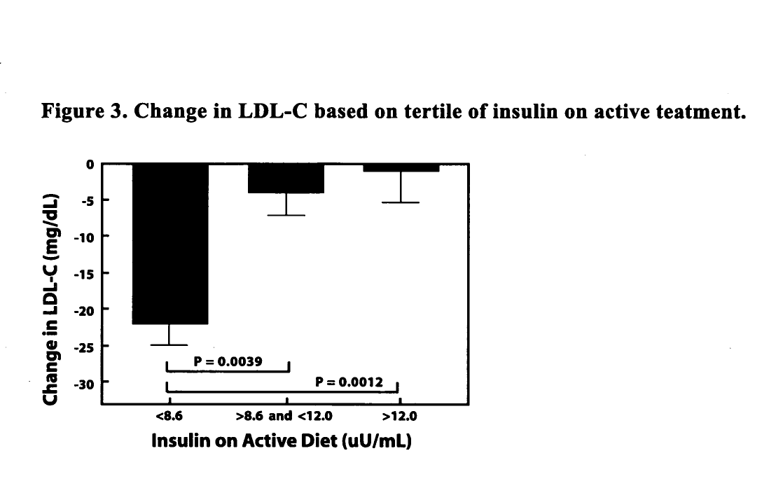

Additionally, all measures of insulin were highly significant predictors of LDL-C response. The best predictor was insulin level measured while on the active treatment (FIG. 2). When the population is split roughly into tertiles based on insulin levels while on active treatment, significant differences in response were found (FIG. 3) with the greatest response for those participants whose insulin levels were at the lower tertile.

This clinical trial is designed to evaluate the efficacy of a nutritional bar in lowering LDL-C, relative to that of a placebo bar, within a controlled average American diet. The nutritional bar contained beta-glucan, soy protein, phytosterols, and omega 3 fatty acids, all known to independently improve markers of CVD risk. The nutritional bar of the invention reduced LDL-C by 8.6 mg/dL relative to the control. Total cholesterol was significantly reduced by 7.7 mg/dL. Participants with higher LDL-C at baseline or who were insulin sensitive had greater reductions than those with lower LDL-C at baseline or who are relatively insulin resistant.

For those individuals who were more insulin sensitive (insulin <8.8 uU/ml) impressive reductions in LDL-C were observed (>20 mg/dL), suggesting that this bar would be highly effective in a large segment of the population.

The examples presented above and the contents of the application define and describe examples of the many cocoa compositions, products, and methods that can be produced or used according to the invention. None of the examples and no part of the description should be taken as a limitation on the scope of the invention as a whole or of the meaning of the following claims.

What is claimed is:

1. A therapeutic or prophylactic method of treating atherosclerosis, thrombosis, heart attack, stroke, or cardiovascular conditions characterized by increased LDL cholesterol levels, comprising administering to a subject in need thereof a composition comprising an effective amount of each of beta-glucan, soy protein, phytosterols, and omega-3 fatty acids, wherein the composition contains about 40/30/30% carbohydrates/protein/fat on an available energy basis,
and wherein the subject maintains a diet that is substantially weight-maintaining on a total calorie basis.

2. The method of claim 1, further comprising monitoring the LDL cholesterol level of the subject.

3. The method of claim 1, wherein the composition possesses a low glycemic index.

4. The method of claim 1, wherein the composition is administered at least twice a day.

5. The method of claim 4, wherein the composition is administered at breakfast and dinner.

6. The method of claim 5, wherein the administration of the composition continues for at least four weeks.

7. The method of claim 6, wherein the LDL cholesterol level of the subject declines by about 5 mg/dl or more.

8. The method of claim 7, wherein the LDL cholesterol level declines by about 8 mg/dl or more.

9. The method of claim 6, wherein the total cholesterol level of the subject declines by about 5 mg/dl or more.

10. The method of claim 9, wherein the total cholesterol level of the subject declines by about 7 mg/dl or more.

11. A nutritional bar or food product or food ingredient comprising a LDL-C reducing effective amount of each of beta glucan, soy protein, phytosterols, and at least one omega-3 fatty acid,
wherein protein is present at about 10-20% by weight, phytosterols are present at about 1 to 2% by weight, omega-3 fatty acids are present at about 1% by weight, and soluble oat fiber is present at about 5 to 6%,
and wherein the bar contains a content of about 40/30/30% carbs/protein/fat on an available energy basis.

12. The nutritional bar or food product of claim 11, further comprising cocoa powder or cocoa liquor.

13. The nutritional bar or food product of claim 11, further comprising fruit or fruit flavor.

14. The nutritional bar or food product of claim 11, further comprising sunflower oil.

15. The nutritional bar or food product of claim 11, further comprising fructose.

16. The nutritional bar or food product of claim 11, further comprising inulin.

17. The nutritional bar or food product of claim 11, further comprising chromium picolinate.

18. The nutritional bar or food product of claim 11, further comprising an antioxidant food ingredient.

19. The nutritional bar or food product of claim 18, wherein the antioxidant food ingredient is a cocoa product.

20. The nutritional bar or food product of claim 18, wherein the antioxidant food product is cinnamon.

21. The nutritional bar or food product of claim 11, wherein the water activity level is about 0.5 or lower.

22. The nutritional bar or food product of claim 19, wherein the water activity level is about 0.5 or lower.

23. The nutritional bar or food product of claim 11, wherein the amount of beta glucan is about 1 to 2 grams, the amount of soy protein is about 10 to 15 grams, the amount of phytosterols is about 0.5 to 1 grams, and the amount of omega-3 fatty acids is about 0.1 to 0.2 grams.

24. The nutritional bar or food product of claim 23, wherein the amount of beta glucan is about 1.5 grams, the amount of soy protein is about 12.5 grams, the amount of phytosterols is about 0.65 grams, and the amount of omega-3 fatty acids is about 0.15 grams.

25. The nutritional bar or food product of claim 23, further comprising a cocoa product.

26. The nutritional bar or food product of claim 24, further comprising a cocoa product.

27. The nutritional bar or food product of claim 23, further comprising cinnamon or a cinnamon flavor.

28. The nutritional bar or food product of claim 24, further comprising cinnamon or a cinnamon flavor.

29. A method of producing a nutritional food product comprising, preparing a dry ingredient mixture comprising soy protein, soluble oat fiber, and dry milk;
preparing a wet mixture comprising liquid fructose (about 90% in water), sunflower oil, phytosterols, and an omega-3 fatty acid;
mixing the dry and wet mixtures together with one or more flavors or flavoring ingredients, and forming the mixture into a final shape and size.

30. The method of claim 29, further comprising enrobing the final shape.

31. The method of claim 29, wherein the flavor or flavoring ingredients comprise a cocoa product.

32. The method of claim 29, wherein the flavor or flavoring ingredients comprise a fruit or fruit product.

33. A method of testing the effect of a food product composition comprising beta glucan, soy protein, phytosterols, and at least one omega-3 fatty acid,
the method comprising orally administering to an individual the food product composition at least once a day, and measuring the status one or more of LDL-C levels, HDL-C levels, triglyceride levels, C-reactive protein, or total cholesterol levels after administration.

34. The method of claim 33, wherein the composition comprises about 10 to 15 grams of soy protein, about 0.6 to 1 grams phytosterols, about 1 to 2 grams soluble oat fiber, and about 0.1 to 0.2 grams omega-3 fatty acids.

35. The method of claim 24, wherein the testing is repeated on a substantially daily basis for a period of at least about two weeks.

36. The nutritional bar or food product of claim 18, wherein the at least one omega-3 fatty acid comprises DHA.

37. The nutritional bar or food product of claim 18, wherein the at least one omega-3 fatty acid comprises EPA.

38. The nutritional bar or food product of claim 18, wherein the at least one omega-3 fatty acid present is substantially DHA.

39. The nutritional bar or food product of claim 18, wherein the at least one omega-3 fatty acids present is substantially EPA.

40. The method of claim 33, further comprising administering one or more compounds to reduce LDL or total cholesterol levels from the group statins, fibrates, resin-based therapies, and ezetimibe.
